(19) United States
(12) Patent Application Publication
(10) Pub. No.: US 2013/0060182 A1
(43) Pub. Date: Mar. 7, 2013

(54) ORTHOTIC TOE SOCK

(71) Applicants: Robert Kotkamaa, Vaasa (FI); Nuutinen Henrik, Mustasaari (FI); Olavi Murros, Helsinki (FI)

(72) Inventors: Robert Kotkamaa, Vaasa (FI); Nuutinen Henrik, Mustasaari (FI); Olavi Murros, Helsinki (FI)

(21) Appl. No.: 13/697,083

(22) PCT Filed: May 5, 2011
(86) PCT No.: PCT/FI11/50413

§ 371 Date: Nov. 9, 2012

(30) Foreign Application Priority Data
May 10, 2010 (FI) 20105508

(51) Int. Cl. A61F 5/00; D04B 39/00; A43B 17/00

(52) U.S. Cl. 602/30; 2/239; 66/178 R

(57) ABSTRACT

The present invention is based on providing, in order to correct or prevent a toe deformity, an orthotic toe sock (S) wherein a toe part (W) of the sock (S) comprises at least two toe-specific toe elements (1, 2, 3, 4, 5) arranged to receive adjacent toes. The adjacent toe elements are, in a corresponding space (I, II, Ill, IV) in between the toes, attached to one another over an area (A) extending, when the sock is being worn, from a base or from above the base, e.g. from a proximal interphalangeal joint region, of the toe in question, e.g. the second, third, fourth or fifth toe, to a tip of the toe or almost to the tip of the toe.

### FIELD OF THE INVENTION

The invention relates to an orthotic toe sock and a method of manufacturing the same.

### BACKGROUND OF THE INVENTION

Hallux valgus refers to a foot problem wherein the big toe turns towards the rest of the toes. At the same time, the metatarsophalangeal joint of the hallux undergoes changes, and the joint as well as the conjoining bursa ache and swell. The changed structure may result in callus formation. The pain may also change the function of the foot and cause pain in the ankle and in the leg.

For the purpose of supporting a slightly turned toe, wedges are available to be positioned between toes for correcting the toe deformity and for reducing ailments. Mild to moderate hallux valgus deformities do not usually benefit from surgical treatment. In a commonly used hallux valgus operation, the first metatarsal bone head is shaped and the joint deformity is thus corrected. After surgery, about 10 to 20% of those who had the operation re-develops the deformity. In order to prevent hallux valgus and to treat mild to moderate forms thereof, it is possible to choose shoes with a low heel and a sufficiently spacious toe box. Toe separators made of silicone set the toes in correct orientation. It is possible to have a between-the-toes support custom-made by a podiatrist. Some patients wear a night splint to stretch the big toe into a correct position. Broken skin may be protected with a foam plastic protection available at pharmacies. A bump area may be protected against pressure by using e.g. silicone orthoses, i.e. toe supports made of silicone, or night splints. It is also possible to reduce pressure on the hallux valgus by choosing shoes that have a sufficiently spacious front part. Foot orthoses sometimes enable abnormal functioning of the foot to be corrected and thus propagation of the deformity to be prevented or delayed.

A problem with the above-described arrangements is that the different supports are difficult to use. Some of them may only be worn at night while sleeping, since when they are worn, it may be difficult to get a shoe on, for instance. It would also be desirable for the supports to stay in place more properly, and they may also cause pain, particularly during walking.

### BRIEF DESCRIPTION OF THE INVENTION

An object of the invention is thus to provide a method and an arrangement so as to enable the aforementioned problems to be solved. The object of the invention is achieved by a method, an orthotic toe sock, and a use that are characterized by what is stated in the independent claims. Preferred embodiments of the invention are disclosed in the dependent claims.

The invention, in order to correct or prevent a toe deformity, is based on providing an orthotic toe sock wherein a toe part of the sock comprises at least two toe-specific toe elements arranged to receive adjacent toes. In a respective space in between the toes, the adjacent toe elements are attached to one another over an area arranged, when the sock is being used, to extend from a base or from above the base of the particular toe almost all the way to a tip of the toe. For instance, if the space in question is the first one in between the toes, the toe in question is the second toe.

An advantage of the method and arrangement according to the invention is that such an orthotic toe sock, when being worn on the foot, stays well in place and is relatively comfortable to use. The orthotic toe sock is easier to use also because therein the orthosis and the sock are provided in the same unit.

### BRIEF DESCRIPTION OF THE FIGURES

The invention is now described in closer detail in connection with preferred embodiments and with reference to the accompanying drawings, in which:

FIG. 1 is a top view illustrating an orthotic toe sock according to a first embodiment of the present solution:

FIG. 2 is a top view illustrating an orthotic toe sock according to a second embodiment of the present solution;

FIG. 3 is a top view illustrating an orthotic toe sock according to a third embodiment of the present solution;

FIG. 4 is a sectional view illustrating an orthotic toe sock according to an embodiment of the present solution during use.

### DETAILED DESCRIPTION OF THE INVENTION

The human hallux or big toe (“the first toe”) has two joints while the other toes, i.e. “the second, third, fourth, and fifth toes” (wherein the fifth toe refers to the little toe), have three joints each. In addition to hallux valgus, toe deformities include conditions like hammer toe, contracted toe, and mallet toe. In the hammer toe deformity, the metatarsophalangeal and distal interphalangeal joints of a toe are extended while the proximal interphalangeal joint of the toe is contracted. In the contracted toe deformity, the metatarsophalangeal joint of a toe is extended while the proximal and distal interphalangeal joints of the toe are contracted. In the mallet toe deformity, the metatarsophalangeal and proximal interphalangeal joints of a toe are in a normal position but the distal interphalangeal joint is in a severely contracted position.

The present solution relates to an orthotic toe sock, i.e. a kind of support sock, which corrects the position of toes. Then, a modified toe sock is used for providing support. An embodiment of the present solution is based on variation in the density of knitting and on the shape of stitches provided in-between the toes, as will be described below.

An orthotic toe sock according to an embodiment of the present solution enables a deformity of one or more toes to be corrected or prevented. A toe part of the orthotic toe sock according to the present solution comprises at least two toe-specific (depending on the number of toes for whose deformity the sock is designed) toe elements arranged to receive adjacent toes such that the adjacent toe elements are in a respective space in between the toes attached to one another over an area arranged, when the sock is being used, to extend from a proximal interphalangeal joint region of a selected toe to the tip or almost to the tip of the toe. It may be thought that the area extends from a distal end of the toe (from the tip or almost from the tip of the toe) to the proximal interphalangeal joint region of the toe in question, or slightly more than proximally. The area may also extend from the base of the toe to the tip or almost to the tip of the toe. If the space in question is the first one in between the toes (i.e. the space in between the big toe and the second toe), the respective adjacent toe elements are in the first space in between the toes attached to one another over an area arranged to extend from the base or from above the base (e.g. from the proximal interphalangeal joint region) of the second toe almost to the tip of the second toe. If the space in question is the second space in between the toes, the respective adjacent toe elements are in the second space in between the toes attached to one another over an area arranged to extend from the base or from above the base (e.g. from the proximal interphalangeal joint region) of the second or third toe almost to the tip of the second or third toe. If the space in question is the third one in between the toes, the respective adjacent toe elements are in the third space in between the toes attached to one another over an area arranged to extend from the base or from above the base (e.g. from the proximal interphalangeal joint region) of the third or fourth toe almost to the tip of the third or fourth toe. If the space in question is the fourth one in between the toes, the respective adjacent toe elements are in the fourth space in between the toes attached to one another over an area arranged to extend from the base or from above the base (e.g. from the proximal interphalangeal joint region) of the fourth or fifth toe almost to the tip of the fourth or fifth toe. Herein, the term “almost to the tip of the toe” may refer e.g. to a region of the “last” (“highest”, closest to the tip), i.e. the proximal interphalangeal joint.

In an orthotic toe sock according to another embodiment of the present solution, the toe elements are attached to one another such that a user's big toe that has turned towards the other toes can be forced into a normal position or closer to the normal position when the sock is being worn on the foot. This enables the problems caused by the user's hallux valgus to be removed or alleviated by means of the orthotic toe sock.

In an orthotic toe sock according to yet another embodiment of the present solution, the toe elements are attached to one another such that the user's hammer toe, mallet toe or contracted toe can be forced into a normal position or closer to the normal position when the sock is being worn on the foot. This enables the problems caused by the user's hammer toe, mallet toe and/or contracted toe to be removed or alleviated by means of the orthotic toe sock.

The toe part of an orthotic toe sock according to still another embodiment of the present solution may be formed from a textile toe sock structure, in which case the adjacent toe elements are attached to one another by using an appropriate elastomer material. The material may comprise e.g. silicone or rubber.

The toe part of an orthotic toe sock of still yet another embodiment of the present solution may be formed from a uniform textile sock structure, in which case the toe elements are formed by knitting the relevant bottom and top loops of the sock together over a desired area (i.e. for instance in the first space in between the toes, over an area arranged to extend from the proximal interphalangeal joint region of the second toe almost to the tip of the second toe, etc.).

The toe part of an orthotic toe sock of still yet another embodiment of the present solution may be formed from a uniform textile sock structure, in which case the toe elements are formed by sewing with flexible stitching the bottom and top of the sock together over a relevant area (i.e. for instance in the first space in between the toes, over an area arranged to extend from the proximal interphalangeal joint region of the second toe almost to the tip of the second toe, etc.).

A thenar part of an orthotic toe sock according to still yet another embodiment of the present solution may comprise a rib part comprising rib knit, the rib part being configured in use to generate a compression effect on the user's thenar. This enables the user's toes to be forced to become separated by making the toes diverge from one another. This enables the user's big toe that has turned towards the other toes to be forced into a normal position or closer to the normal position by means of the orthotic toe sock. Such a sock is useful particularly as a “postoperative sock” to be used after a hallux valgus operation.

A plantar arch part of an orthotic toe sock according to still yet another embodiment of the present solution comprises a rib part comprising rib knit, the rib part being configured in use to generate a compression effect on the user's plantar arch. This enables the user's big toe that has turned towards the other toes to be forced into a normal position or closer to the normal position by means of the orthotic toe sock. Such a sock is useful e.g. as a “commercial sock” to be used for preventing hallux valgus. The commercial sock may also be implemented such that it receives the fourth and fifth toes in the same toe element, i.e. the sock is provided with one common toe element for the fourth and fifth toes, if the purpose is not to prevent/correct the deformity of the fourth/fifth toe. This makes the sock easier to put on e.g. for those who have a predisposition to developing hallux valgus but have no problems with the fourth/fifth toes.

The aforementioned rib part may in a width direction of the sock extend around the sock, and in a length direction of the sock it may be e.g. 1 to 15 cm long. The rib part of the postoperative sock may be longer (e.g. about 12 cm) than the rib part of the commercial sock (e.g. about 5 cm). Instead of or in addition to rib, it is feasible to use a corresponding tapered part in the sock in order to generate a compression effect on the thenar and/or the plantar arch.

An orthotic toe sock according to yet another further embodiment of the present solution may be shaped to suit the right and/or the left foot. It may, for instance, comprise a part which is shaped to receive the user's heel. On the other hand, the sock may be suitable for use on both the left and the right foot. An orthotic toe sock according to an embodiment of the present solution may be shaped for receiving the toes such that each toe element tightly surrounds the respective toe. It is also possible to shape the orthotic toe sock user-specifically. For instance, the area of a space in between the toes (i.e. for example the common loops between the bottom and top of the sock, flexible stitching forming the toe elements, or an area of silicone or another elastomer material for connecting separate toe elements (which may also be called a seam)) over which adjacent toe elements are attached to one another may be dimensioned according to the needs of a user (e.g. according to a distance between the proximal interphalangeal joint of a toe and the base of the toe, or the length and/or width of the user's toes in the first place).

An orthotic toe sock according to yet another further embodiment of the present solution may comprise an ankle sock (or a calf sock), a knee-high sock, a sleeveless sock, a long sock or pantyhose, i.e. tights, and it may be made from yarn comprising e.g. elastane, cotton, polyamide, polyester, wool, silk, polyacryl, linen and/or bamboo fibre and/or a mixture thereof or any other textile yarn.

An orthotic toe sock according to yet another further embodiment of the present solution may be weft-knitted or warp-knitted by a knitting machine (e.g. by a flat knitting machine, a circular knitting machine, a 3D flat knitting machine (Shima Seiki, Stoll or the like)). Herein, warp knitting refers to making a crocheting-like knitted fabric while weft knitting refers to making a textile resembling an ordinary knitted fabric. The knitting machines used may be ones designed for industrial and/or domestic use.

A method according to yet a further embodiment of the present solution for manufacturing an orthotic toe sock comprises forming a sock toe part comprising at least two toe-specific toe elements arranged to receive adjacent toes, the adjacent toe elements being in a respective space in between the toes attached to one another over an area arranged to extend from a proximal interphalangeal joint region (or from the base/from above the base) of a selected toe to the tip or almost to the tip of the toe. If the space in question is the second one in between the toes, the corresponding adjacent toe elements are in the second space in between the toes attached to one another over an area arranged to extend from a proximal interphalangeal joint region (or from the base/or from above the base) of the second or third toe almost to the tip of the second or third toe. If the space in question is the third one in between the toes, the corresponding adjacent toe elements are in the third space in between the toes attached to one another over an area arranged to extend from the proximal interphalangeal joint region (or from the base/or from above the base) of the third or fourth toe almost to the tip of the third or fourth toe. If the space in question is the fourth one in between the toes, the corresponding adjacent toe elements are in the fourth space in between the toes attached to one another over an area arranged to extend from the proximal interphalangeal joint region (or from the base/or from above the base) of the fourth or fifth toe almost to the tip of the fourth or fifth toe. Herein, the term “almost to the tip of the toe” may refer to a region of the last joint (residing closest to the tip) of the particular toe.

A method according to still yet a further embodiment of the present solution for manufacturing an orthotic toe sock comprises forming the sock toe part by forming a textile toe sock structure and by attaching adjacent toe elements to one another by using an appropriate elastomer material for forcing a toe into a normal position or closer to the normal position when the sock is being worn.

A method according to still yet another embodiment of the present solution for manufacturing an orthotic toe sock comprises forming the sock toe part by forming a uniform textile sock structure and by forming the toe elements by weft knitting the respective bottom and top loops of the sock together over a desired area in order to force a toe into a normal position or closer to the normal position when the sock is being worn.

A method according to still yet another embodiment of the present solution for manufacturing an orthotic toe sock comprises forming the sock toe part by forming a uniform textile sock structure and by forming the toe elements by sewing with flexible stitching the bottom and top of the sock together over a respective area in order to force a toe into a normal position or closer to the normal position when the sock is being worn.

A method according to still yet another further embodiment of the present solution for manufacturing an orthotic toe sock comprises forming a thenar part of the sock by forming a rib part comprising rib knit, the rib part being configured to generate a compression effect on the user's thenar when the sock is being worn.

A method according to still yet another further embodiment of the present solution for manufacturing an orthotic toe sock comprises forming a plantar arch part of the sock by forming a rib part comprising rib knit, the rib part being configured to generate a compression effect on the user's plantar arch when the sock is being worn.

A method according to still yet another further embodiment of the present solution for manufacturing an orthotic toe sock comprises weft knitting or warp knitting the orthotic toe sock by a knitting machine.

An orthotic toe sock according to still another embodiment of the present solution is suitable for use in treatment or prevention e.g. of hallux valgus, hammer toe, mallet toe, contracted toe and/or callosity.

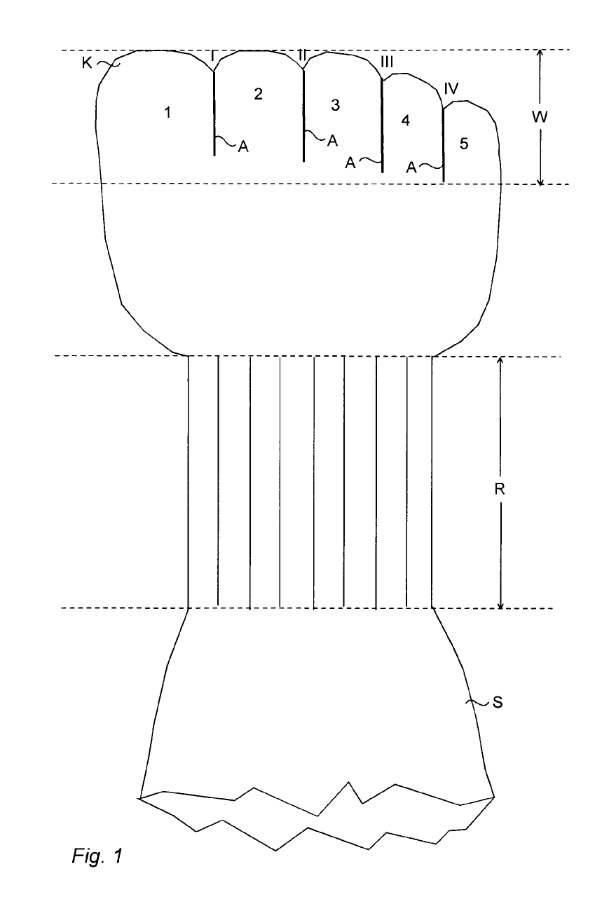

FIG. 1 shows an orthotic toe sock according to a first embodiment of the present solution. Referring to FIG. 1, the orthotic toe sock S according to the first embodiment of the present solution comprises five toe-specific toe elements 1, 2, 3, 4, 5 arranged to receive adjacent toes such that the adjacent toe elements (i.e. 1+2, 2+3, 3+4, 4+5) are in the corresponding space I, II, III, IV in between the toes attached to one another over an area A arranged, when the sock is being worn, to extend from a proximal interphalangeal joint region (or from the base/from “above” the base) of a selected toe almost to the tip (or to the tip) of the toe. In the situation shown in FIG. 1, a toe part W of the sock is formed from a uniform textile sock structure, in which case the toe elements 1, 2, 3, 4, 5 are formed by knitting the respective loops of the bottom and top of the sock together over the desired area A. The sock according to FIG. 1 may be made e.g. by weft knitting. For the purpose of receiving the tips of the toes, tips K of the toe elements 1, 2, 3, 4, 5 may be knitted e.g. by suitably decreasing and casting off loops so as to form the tip part K of the sock S. A thenar part and/or a plantar arch part of the sock may comprise a rib part R comprising rib knit, the rib part R being configured to generate during use a compression effect on the user's thenar/plantar ach, respectively. In a width direction of the sock S the rib part R extends around the sock, and in a length direction of the sock S it may preferably be about 1 to 15 cm long, more preferably about 5 to 12 cm long.

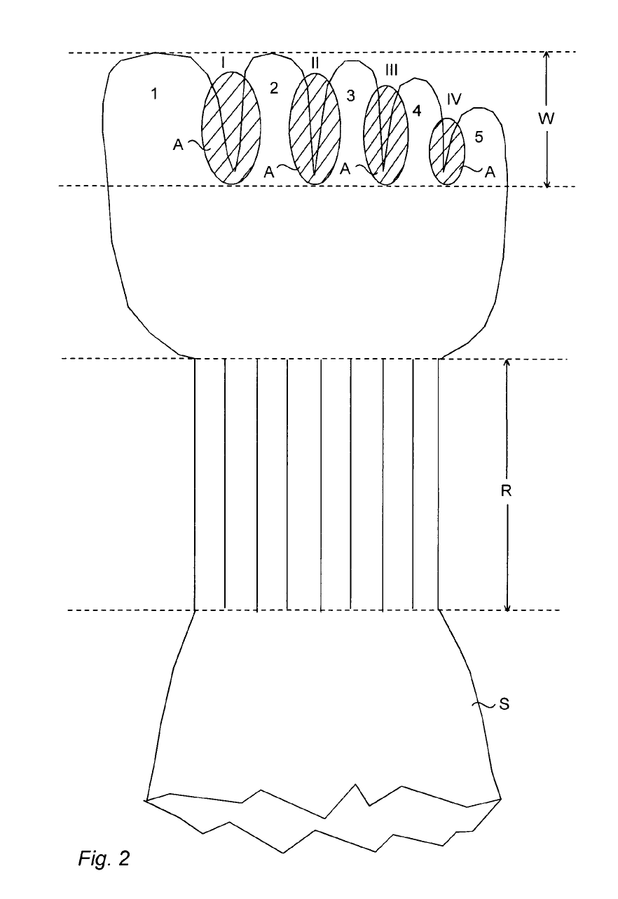

FIG. 2 shows an orthotic toe sock according to a second embodiment of the present solution. Referring to FIG. 2, the orthotic toe sock S according to the second embodiment of the present solution comprises five toe-specific toe elements 1, 2, 3, 4, 5 arranged to receive adjacent toes such that the adjacent toe elements (i.e. 1+2, 2+3, 3+4, 4+5) are in the corresponding space I, II, Ill, IV in between the toes attached to one another over an area A arranged, when the sock is being worn, to extend from a proximal interphalangeal joint region (or from the base/from above the base) of a selected toe almost to the tip (or to the tip) of the toe. In the situation shown in FIG. 2, the toe part W of the sock is formed from a textile toe sock structure, in which case the adjacent toe elements 1, 2, 3, 4, 5 are attached to one another by using a suitable elastomer or thermoplastic material, such as silicone or an appropriate rubber or plastic. This may be achieved e.g. by a suitable seaming technology. The elastomer or thermoplastic material may also be spread on the sock S e.g. over an entire or an almost entire toe part W rather than only in-between the toes, as shown in FIG. 2. The material may be spread e.g. by brushing, spraying or dipping. The sock according to FIG. 2 may be made e.g. by warp knitting. The thenar part and/or the plantar arch part of the sock may comprise a rib part R comprising rib knit, the rib part R being configured to generate during use a compression effect on the user's thenar/plantar ach, respectively. In the width direction of the sock S the rib part R extends around the sock, and in the length direction of the sock S it may preferably be about 1 to 15 cm long (more preferably about 5 to 12 cm long).

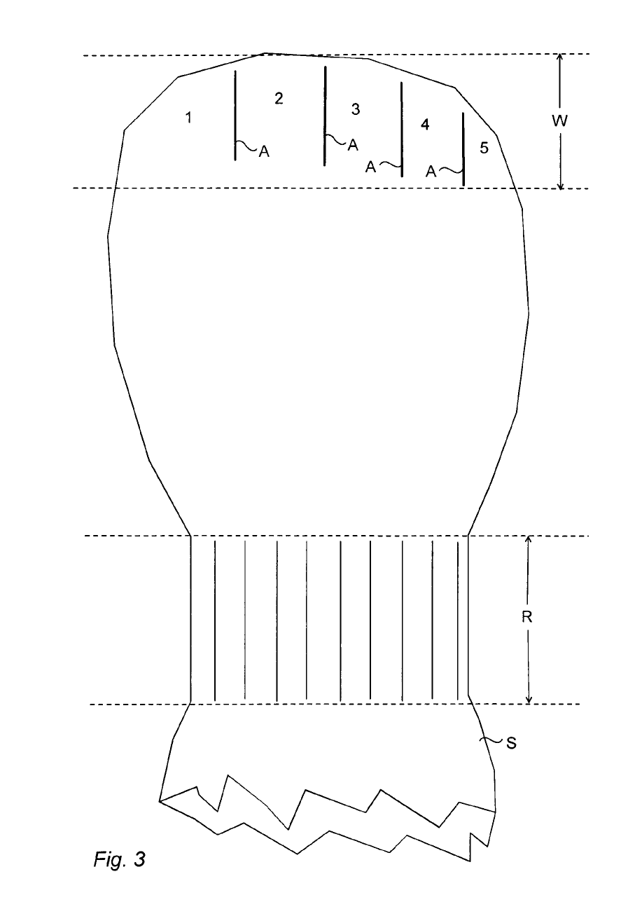

FIG. 3 shows an orthotic toe sock according to a third embodiment of the present solution. Referring to FIG. 3, the orthotic toe sock S according to the third embodiment of the present solution comprises five toe-specific toe elements 1, 2, 3, 4, 5 arranged to receive adjacent toes such that the adjacent toe elements (i.e. 1+2, 2+3, 3+4, 4+5) are in the corresponding space I, II, III, IV in between the toes attached to one another over an area A arranged, when the sock is being worn, to extend from a proximal interphalangeal joint region (or from the base/from above the base) of a selected toe almost to the tip (or to the tip) of the toe. In the situation shown in FIG. 3, the toe part W of the sock is formed from a uniform textile sock structure, in which case the toe elements 1, 2, 3, 4, 5 are formed by sewing with flexible stitch (stitching) the bottom and top of the sock together over the respective area A. The sock according to FIG. 3 may be made e.g. by weft knitting or warp knitting. The sewing may be carried out e.g. by a looping machine or a chain stitch machine. The thenar part and/or the plantar arch part of the sock may comprise a rib part R comprising rib knit, the rib part R being configured to generate during use a compression effect on the user's thenar/plantar arch, respectively. In the width direction of the sock S the rib part R extends around the sock, and in the length direction of the sock S it may preferably be about 1 to 15 cm long, more preferably about 5 to 12 cm long. Alternatively, such an orthotic toe sock may be made from a toe sock type structure wherein the toe elements are arranged to extend from the tip to the proximal interphalangeal joint region, in which case the adjacent toe elements are attached together by sewing over the appropriate area in-between the toes (i.e. from the proximal interphalangeal joint region to the tip or almost to the tip) (not shown in FIG. 3).

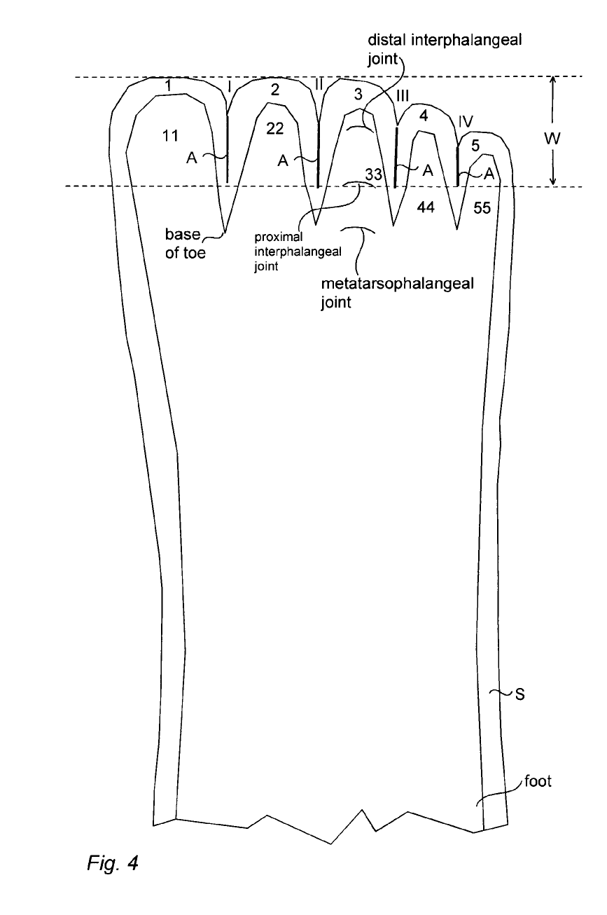

FIG. 4 illustrates an orthotic toe sock according to an embodiment of the present solution during use (i.e. when the sock S is worn on a foot J). Referring to FIG. 4, the orthotic toe sock S according to an embodiment of the present solution comprises five toe-specific toe elements 1, 2, 3, 4, 5 arranged to receive adjacent toes 11, 22, 33, 44, 55 such that the adjacent toe elements (i.e. 1+2, 2+3, 3+4, 4+5) are in the corresponding space I, II, III, IV in between the toes attached to one another over an area A arranged, when the sock is being worn, to extend from a proximal interphalangeal joint region (or from the base/from above the base) of the selected toe 22, 33, 44, 55 almost to the tip (or to the tip) of the toe 22, 33, 44, 55. Consequently, the area A does not extend all the way to the base/metatarsophalangeal joint of the toes 11, 22, 33, 44, 55. The area A may extend e.g. somewhere near the distal interphalangeal joint of the toe 22, 33, 44, 55.

If the sock is to be worn by a user missing a toe/toes, a toe element corresponding with such a missing toe may be omitted, and toe elements adjacent thereto are not attached to one another. For instance, if the third toe is missing, the third toe element may be omitted from the sock, in which case the second and the fourth toe elements are not attached to one another. Similarly, if a user has excessive toes, the sock may be provided with a corresponding number of excessive toe elements.

An orthotic toe sock according to the present solution may also be implemented such that the toe elements are left open at the tips.

An orthotic toe sock according to a still further embodiment of the present solution may be implemented as a postoperative sock for professional use (e.g. for use after a hallux valgus operation), in which case the sock is typically manufactured according to the user's measurements. It may then be most appropriate to utilize a sock wherein separate toe elements are attached to one another in the above-described manner with silicone or a corresponding substance. The sock may also be implemented as a more commercial version, in which case the same sock size fits a plurality of users. In such a case, it is for instance possible to manufacture the sock in the above-described manner as a uniform structure by weft knitting, whereby the toe elements are already in the knitting phase formed by knitting certain bottom and top loops together in order to form the toe elements.

It is to be noted that an ordinary (glove-like) toe sock does not enable the sock to be used for treatment or prevention of hallux valgus, hammer toe, mallet toe, contracted toe and/or callosity since movement of the toes with respect to one another does not change much as compared with an ordinary sock. Consequently, when using the ordinary toe sock, the big toe of a patient suffering e.g. from hallux valgus is allowed to turn freely into an incorrect position, i.e. towards the other toes. When, instead, using an orthotic toe sock according to the present solution, it is possible to guide the toes into the correct position as efficiently as possible when the toe elements are not in a glove-like manner separate from one another but are in-between the toes arranged to be attached to one another by gluing, sewing or knitting near to the middle of a toe. A toe element does not necessarily extend to the base of the toe but may start above the base, e.g. near the proximal interphalangeal joint of the user's toe. When using a sock according to the present solution, the rest of the toes in line serve to support the problem toe(s).

The orthotic toe sock according to the present solution is easy and inexpensive to manufacture. For instance in the case of a weft-knitted sock, the entire sock may be made by a weft knitting machine at one go in a couple of minutes. In such a case, a manufacturing process is started from the mouth part of the sock, which provides a tubular structure (which may be provided with the above-described rib part). As the manufacturing progresses to the toe part, the knitting machine is configured to further knit the toe part as a uniform structure such that the respective bottom and top loops are knitted together in order to form the toe elements. Next, the tips of the toe parts are knitted for receiving the tips of the toes e.g. by appropriately decreasing and casting off loops. The knitting machine may be an electronic programmable knitting machine, in which case it is programmable to manufacture an orthotic toe sock according to the present solution.

In the sock according to the present solution, operative parts, such as the toe elements, the toe part and/or the rib part, may be knitted/crocheted in different colours, which makes it easier for the user to choose an orthotic toe sock suitable for his or her foot shape and put it on such that the parts settle appropriately on the foot.

It is obvious to one skilled in the art that as technology advances, the basic idea of the invention may be implemented in many different ways. The invention and its embodiments are thus not restricted to the above-described examples but may vary within the scope of the claims.

1. An orthotic toe sock for correcting or preventing a deformity of one or more toes, wherein
a toe part of the sock comprises at least two toe-specific toe elements arranged to receive adjacent toes such that the adjacent toe elements are, in a corresponding space in between the toes, attached to one another over an area arranged to extend, when the sock is being worn on a foot, from a proximal interphalangeal region of a selected toe almost to the tip of the toe, whereby if the space in between the toes in question is the first one, the selected toe is the second toe.

2. (canceled)

3. An orthotic toe sock as claimed in claim 1, wherein if the space in between the toes in question is the second, third or fourth one, the selected toe is the second, third, fourth or fifth toe, respectively.

4. An orthotic toe sock as claimed in claim 1, wherein the toe elements are attached to one another such that a user's hallux turned towards the other toes is forced into a normal position or closer to the normal position when the sock is being worn on the foot.

5. An orthotic toe sock as claimed in claim 1, wherein the toe elements are attached to one another such that the user's hammer toe, mallet toe or contracted toe is forced into the normal position or closer to the normal position when the sock is being worn on the foot.

6. An orthotic toe sock as claimed in claim 1, wherein the toe part of the sock is formed from a textile toe-sock-like structure, whereby the adjacent toe elements are attached to one another by using an appropriate elastomer material.

7. An orthotic toe sock as claimed in claim 6, wherein the elastomer material comprises silicone.

8. An orthotic toe sock as claimed in claim 1, wherein the toe part of the sock is formed from a uniform textile sock structure, whereby the toe elements are formed by knitting respective bottom and top loops of the sock together over a desired area.

9. An orthotic toe sock as claimed in claim 1, wherein the toe part of the sock is formed from a uniform textile sock structure, whereby the toe elements are formed by sewing with flexible stitching a bottom and a top of the sock together over the respective area.

10. An orthotic toe sock as claimed in claim 1, wherein the toe part of the sock is formed from a textile toe-sock-like structure, whereby adjacent toe elements are attached to one another by sewing with flexible stitching the adjacent toe elements together over the respective area.

11. An orthotic toe sock as claimed in claim 1, wherein a thenar part of the sock comprises a rib part comprising rib knit, the rib part being configured to generate during use a compression effect on the user's thenar.

12. An orthotic toe sock as claimed in claim 1, wherein a plantar arch part of the sock comprises a rib part comprising rib knit, the rib part being configured to generate during use a compression effect on the user's plantar arch.

13. An orthotic toe sock as claimed in claim 11, wherein the rib part in a width direction of the sock extends around the sock and in a length direction of the sock is preferably about 1 to 15 cm long, more preferably about 5 to 12 cm long.

14. An orthotic toe sock as claimed in claim 1, wherein it is shaped to suit at least one of the right and/or the left foot.

15. An orthotic toe sock as claimed in claim 1, wherein it is shaped to receive toes such that each toe element is arranged to tightly surround a corresponding toe when the sock is being used.

16. An orthotic toe sock as claimed in claim 1, wherein it is shaped user-specifically.

17. An orthotic toe sock as claimed in claim 1, wherein it comprises an ankle sock, a knee-high sock, a sleeveless sock, a long sock or a pantyhose.

18. An orthotic toe sock as claimed in claim 1, wherein it is made from yarn comprising at least one of elastane, cotton, polyamide, polyester, wool, silk, polyacryl, linen and/or bamboo fibre.

19. An orthotic toe sock as claimed in claim 1, wherein it is weft-knitted by a knitting machine.

20. An orthotic toe sock as claimed in claim 1, wherein it is warp-knitted by a knitting machine.

21. A method for manufacturing an orthotic toe sock for correcting or preventing a deformity of one or more toes, the method comprising
forming, by knitting by a knitting machine, a sock toe part comprising at least two toe-specific toe elements arranged to receive adjacent toes,
adjacent toe elements being, in a corresponding space in between the toes, attached to one another over an area arranged to extend, when the sock is being worn on a foot, from a proximal interphalangeal joint region of a selected toe to a tip of the toe or almost to the tip of the toe,
whereby if the space in between the toes in question is the first one, the selected toe is the second toe,
the method comprising
forming the sock toe part by forming a uniform textile sock structure and by forming during a knitting phase the toe elements by weft knitting respective bottom and top loops of the sock together over a desired area in order to force a toe into a normal position or closer to the normal position when the sock is being worn, or
forming the sock toe part by forming a textile toe-sock-like structure and attaching adjacent toe elements to one another by using an appropriate elastomer material in order to force the toe into a normal position or closer to the normal position when the sock is being worn.

22. (canceled)

23. A method as claimed in claim 21, wherein if the space in between the toes in question is the second, third or fourth one, the selected toe is the second, third, fourth or fifth toe, respectively.

24. A method as claimed in claim 21 wherein elastomer material comprises silicone.

25. A method as claimed in claim 21, wherein the method comprises forming, by knitting by a knitting machine, a thenar part of the sock by forming a rib part comprising rib knit, the rib part being configured to generate a compression effect on the user's thenar when the sock is being worn.

26. A method as claimed in claim 21, wherein the method comprises forming, by knitting by a knitting machine, a plantar arch part of the sock by forming a rib part comprising rib knit, the rib part being configured to generate a compression effect on the user's plantar arch when the sock is being worn.

27. A method as claimed in claim 21, wherein the method comprises shaping an orthotic toe sock to suit at least one of the right and the left foot.

28. A method as claimed in claim 21, wherein the method comprises shaping an orthotic toe sock user-specifically.

29. A method as claimed in claim 21, wherein the method comprises weft-knitting an orthotic toe sock by a knitting machine.

30. A method as claimed in claim 21, wherein the method comprises warp-knitting an orthotic toe sock by a knitting machine.
